(19) United States
(12) Patent Application Publication
(10) Pub. No.: US 2026/0224900 A1
(43) Pub. Date: Aug. 6, 2026

(54) IMPLANTABLE MEDICAL DEVICE WITH IMPROVED LONG-TERM MODE APPLIED IN CASE OF ATRIAL TACHYCARDIA

(71) Applicant: BIOTRONIK SE & Co., KG, Berlin (DE)

(72) Inventors: Frank BECKER, Berlin (DE); Tobias THEILER, Goldswil b. Interlaken (CH)

(73) Assignee: BIOTRONIK SE & Co., KG, Berlin (DE)

(21) Appl. No.: 19/150,872

(22) PCT Filed: Feb. 1, 2024
(86) PCT No.: PCT/EP2024/052508

§ 371 Date: Jul. 24, 2025

(30) Foreign Application Priority Data
Feb. 7, 2023 (EP) 23155343.9

(51) Int. Cl. A61N 1/368; A61B 5/352; A61B 5/353; A61N 1/362

(52) U.S. Cl. CPC A61N 1/3688; A61B 5/352; A61B 5/353; A61N 1/3624

(57) ABSTRACT

Implantable medical device for stimulating a heart, including a processor, a memory unit, a stimulation unit configured to stimulate a heart, a first detection unit configured to detect an electric signal in an atrium of the heart, and a second detection unit configured to detect an electric signal in a ventricle of the heart. In operation, the device: repeatedly detects an atrial electric signal; repeatedly determines whether the atrial electric signal is indicative of atrial fibrillation; if the atrial electric signal is indicative of atrial fibrillation, switch an operational mode of the device from a regular mode to a fibrillation mode, wherein the fibrillation mode allows a ventricular controlled stimulation of a ventricle of the heart; and if the fibrillation mode has been active for a predetermined period of time, switch the operational mode of the device from the fibrillation mode to an atrial-fibrillation optimized long-term mode.

### CROSS-REFERENCE TO RELATED APPLICATIONS

This application is the United States National Phase under 35 U.S.C. § 371 of PCT International Patent Application No. PCT/EP2024/052508, filed on Feb. 1, 2024, which claims the benefit of European Patent Application No. 23155343.9, filed on Feb. 7, 2023, the disclosures of which are hereby incorporated by reference herein in their entireties.

### TECHNICAL FIELD

The present invention relates to an implantable medical device for stimulating a human or animal heart according to the preamble of claim 1, to a method for controlling such an implantable medical device according to the preamble of claim 12, and to a computer program product according to the preamble of claim 15.

### BACKGROUND

Implantable medical devices for stimulating a human or animal heart, such as pacemakers, have been known for a long time. They can perform different functions. Different stimulation programs can be carried out by an appropriate pacemaker to restore the treated heart to a normal state.

Paroxysmal atrial fibrillation often degenerates to long lasting or even chronic atrial fibrillation (AF). In implantable cardiac pacemakers known from prior art, the detection of such an atrial fibrillation serves for a temporarily adjustment of the operational mode of the cardiac pacemaker due to the applied mode switch algorithm. Atrial based operational modes (such as dual pacing, dual detection, and dual control mode, DDD; or ventricular pacing, dual detection, and dual control mode, VDD) are switched to ventricular based operational modes (such as dual pacing, dual sensing, and an inhibiting control mode, DDI; or ventricular pacing, ventricular sensing and an inhibiting control mode, VVI).

In this context, the mode switch algorithm is appropriate to automatically react on paroxysmal atrial fibrillation. However, the applied mode switching mode is not designed to provide patients with a therapeutic pacing over an extended period of time if the patient remains for a longer time in a state of atrial fibrillation. One of the main problems of the applied mode switch mode is that different functions and algorithms that may have a positive impact on the health status of the patient are temporarily deactivated (suspended) in the mode switch mode. They can only be reactivated during a follow-up by manual interaction from a medical doctor.

### SUMMARY

It is an object of the present invention to provide an implantable medical device for stimulating the human or animal heart that overcomes the shortcomings of prior art and that allows better therapy of patients remaining for an extended period of time in a state of an atrial tachycardia, in particular atrial fibrillation.

This object is achieved with an implantable medical device for stimulating the human or animal heart having the features explained in the following. Such an implantable medical device comprises a processor, a memory unit, a stimulation unit, a first detection unit, and a second detection unit. The stimulation unit serves for stimulating a human or animal heart. The first detection unit serves for detecting an electric signal in an atrium of this heart. The second detection unit serves for detecting an electric signal in a ventricle of the heart.

According to an aspect of the presently claimed invention, the memory unit comprises a computer-readable program that causes the processor to perform the steps explained in the following when executed on the processor.

In order to determine the status of an atrium, in particular of the right atrium, of the heart to be stimulated, an atrial electric signal is repeatedly detected with the first detection unit in one of the method steps.

Based on the detected atrial signal, it is repeatedly determined whether an atrial fibrillation is present. Such determination can be easily done, in an embodiment, on the basis of the rate of the detected atrial signal. If the rate exceeds a threshold lying in a range of from 180 beats per minute (bpm) to 300 bpm, in particular of from 190 bpm to 290 bpm, in particular of from 200 bpm to 280 bpm, in particular of from 210 bpm to 270 bpm, in particular of from 220 bpm to 260 bpm, in particular of from 230 bpm to 250 bpm, the detected atrial electric signal is considered to be indicative for an atrial fibrillation.

If the atrial electric signal has been determined to be indicative for an atrial fibrillation, an operational mode of the implantable medical device is switched from a regular mode to an atrial fibrillation mode. In this context, the atrial fibrillation mode exclusively allows a ventricular controlled stimulation of the ventricle of the heart to be stimulated. At the same time, at least one additional function is deactivated.

Said additional function uses information from the right atrium which is not in an AF state, as for instance whether an atrioventricular sequence has been paced or sensed. At the time an AF occurs in the right atrium and when a mode switch is performed from atrial controlled to ventricular controlled stimulation of the ventricle, at least one of the additional functions is therefore deactivated, since no pacing or sensing information except for AF is available.

In particular, the at least one additional function may be chosen from the group comprising, in particular consisting of automatic pacing threshold monitoring, automatic sensing threshold monitoring, closed loop stimulation, beat-to-beat continuous capture control and/or automatic CRT (Cardiac Resynchronization Therapy)-stimulation adaption.

In the further course of the method applied by the implantable medical device, it is evaluated whether a switching criterion is fulfilled. In particular, the switching criterion may be at least one of the following four criteria. The first criterion is fulfilled if the atrial electric signal indicative for atrial fibrillation is detected over a time period of at least 15 minutes, in particular over a time period lying within a range of from 15 minutes to 30 minutes, from 30 minutes to 1 hour, from 1 hour to 12 hours, in particular of from 1.5 hours to 11 hours, in particular of from 2 hours to 10 hours, in particular 5 hours, in particular of from 2.5 hours to 9 hours, in particular of from 3 hours to 8 hours, in particular of from 3.5 hours to 7 hours, in particular of from 4 hours to 6 hours, in particular of from 4.5 hours to 5 hours. In particular, a time period of at least 30 minutes is preferable for the detection of an atrial electric signal indicative of atrial fibrillation. This first criterion is an AF-based criterion which is independent on the operational mode of the implantable medical device.

According to the research underlying the present invention, it has been identified that the duration of atrial fibrillations occur among the affected population in a broad range from seconds to several hours. In order to achieve a positive therapeutic effect for a high number of patients on the one hand, and efficient usage of computational resources on the other hand by avoiding even atrial fibrillations with short durations lead to a mode switch (which would require a mode switch back to an atrial controlled stimulation of the ventricle shortly afterwards), it has been determined that the above mentioned detection times for fulfilling the first criterion represent a suitable tradeoff. In particular, detection times from 15 minutes to 1 hour, and especially 30 minutes, showed effective results.

The second possible criterion is fulfilled if the atrial fibrillation mode is active over time period of at least one hour, in particular over a time period lying within a range of from 15 minutes to 1 hour, from 1 hour to 12 hours, in particular of from 1.5 hours to 11 hours, in particular 6 hours, in particular of from 2 hours to 10 hours, in particular of from 2.5 hours to 9 hours, in particular of from 3 hours to 8 hours, in particular of from 3.5 hours to 7 hours, in particular of from 4 hours to 6 hours, in particular of from 4.5 hours to 5 hours. Preferably, the time period in which the atrial fibrillation mode is active to fulfill the second criterion is 30 minutes. A time period of 30 minutes represents an effective tradeoff between achieving a positive therapeutic effect for a high number of patients on the one hand, and efficient usage of computational resources on the other hand. The second criterion is an AF-independent criterion that is based on the operational mode of the implantable medical device.

The third criterion is fulfilled if the atrial fibrillation mode is active over a cumulative time period of at least 2 hours within 24 hours, in particular over a cumulative time period lying in a range of from 2 hours to 12 hours, in particular of from 3 hours to 11 hours, in particular 8 hours, in particular of from 4 hours to 10 hours, in particular of from 5 hours to 9 hours, in particular from 6 hours to 8 hours, within 24 hours. This third criterion is once again an AF-independent criterion that is based on the operational mode of the implantable medical device.

The fourth criterion is fulfilled if the atrial fibrillation mode has been activated at least 120 times within 24 hours, in particular at least 130 times within 24 hours, in particular at least 140 times within 24 hours, in particular at least 150 times within 24 hours, in particular at least 160 times within 24 hours, in particular at least 170 times within 24 hours, in particular at least 180 times within 24 hours, in particular at least 190 times within 24 hours, in particular at least 200 times within 24 hours.

If the switching criterion is fulfilled, this indicates that the patient suffers from a long lasting atrial fibrillation. In such a case, the operational mode of the implantable medical device is switched from the atrial fibrillation mode to an atrial-fibrillation optimized long-term mode. This atrial-fibrillation optimized long-term mode exclusively allows a ventricular controlled stimulation of the ventricle of the heart to be stimulated. At the same time, the at least one additional function, which, in particular, may be chosen from the group comprising, in particular consisting of, automatic pacing threshold monitoring, automatic sensing threshold monitoring, closed loop stimulation, beat-to-beat continuous capture control and/or automatic CRT (Cardiac Resynchronization Therapy)-stimulation adaption, that was deactivated in the atrial fibrillation mode is reactivated. According to an embodiment of the present invention, closed loop stimulation is understood as electrical stimulation with a pacing rate adapted to a measured cardiac impedance. Thus, the atrial-fibrillation optimized long-term mode is intended to be applied over a longer period of time. For this purpose, functions that are deactivated in the atrial fibrillation mode but that are considered to have a particular positive impact for the patient are automatically reactivated so as to offer the patient a more appropriate therapy providing higher functionality than the limited atrial fibrillation mode. Whereas the atrial fibrillation mode is similar to the mode switch mode of prior art, an automatic switch to an atrial-fibrillation optimized long-term is not known from prior art. Due to the reactivated functions that had been deactivated in the atrial fibrillation mode, the atrial-fibrillation optimized long-term mode is particularly appropriate to provide the patient with the required pacing over an extended period of time during which the patient suffers from an atrial fibrillation. The meaning of reactivation in the context of the invention comprises that the functions are adapted to the ventricular controlled stimulation mode such that they will in the further operation only use the information available within the ventricular controlled stimulation mode.

The method explained in the preceding paragraphs efficiently prevents a long lasting suspension of relevant or important functions of the implantable medical device due to a long lasting atrial fibrillation. By automatically detecting the atrial cardiac state of the heart to be stimulated and by choosing a particularly appropriate operational mode of the implantable medical device to stimulate the heart in the detected atrial cardiac state, a particularly appropriate pacing is made possible by the claimed implantable medical device. The implantable medical device allows a much simpler programming of the implantable medical device and does not require an interaction with medical staff in order to reprogram the implantable medical device after having detected a state of atrial fibrillation. Thus, the operational mode of the implantable medical device is much more quickly adjusted to the medical needs of the patient carrying the device, wherein an automatic switch of the operational modes in dependence on the factual atrial cardiac state of the heart to be stimulated is made possible.

To make it possible for the implantable medical device to terminate the actual atrial-fibrillation optimized long-term mode if it is considered to be no longer necessary from a physiologic point of view, the method comprises, in an embodiment, the further step explained in the following. This step is performed after the step of switching the operational mode of the implantable medical device from the atrial fibrillation mode to the atrial-fibrillation optimized long-term mode.

It is determined if one of the two following criteria is fulfilled. The first criterion is fulfilled if the atrial electric signal is no longer indicative for an atrial fibrillation. This criterion is an AF-dependent criterion that is independent on the operational mode of the implantable medical device.

The second criterion is fulfilled if the atrial electric signal has not been indicative for atrial fibrillation over a time period of at least 1 minute, in particular over a time period lying in a range of from 1 minute to 30 minutes, in particular of from 1.5 minutes to 25 minutes, in particular 15 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes, preferably 15 minutes. This second criterion is also an AF-dependent criterion that is independent on the operational mode of the implantable medical device.

If one of the precedingly explained criteria is fulfilled, the operational mode of the implantable medical device is switched from the atrial-fibrillation optimized long-term mode to a confirmation phase. This confirmation phase serves for an adequate pacing strategy during a period in which the patient does no longer suffer from atrial fibrillation but still has an atrial tachycardia, i.e. an atrial rate that is higher than the regular atrial rate. In the confirmation phase, an exclusively ventricular controlled stimulation of the ventricle of the heart to be stimulated is allowed. At the same time, the confirmation phase comprises activated additional functions that correspond to the activated additional functions of the atrial-fibrillation optimized long-term mode. The confirmation phase serves as transition state in which an evaluation is conducted by the implantable device to determine whether a switch of the implantable device to the regular mode, i.e. the atrial controlled stimulation mode, can be performed.

In an embodiment, the evaluate comprises an evaluation if among the last Y number of heart cycles, an X number of heart cycles occurred with an atrial heart rate below a threshold rate. For instance, the implantable device may evaluate whether 3 out of 5 cardiac intervals have an atrial rate need to be below a threshold rate of 160-200 bpm, in particular 180 bpm.

In a further method step employed in an embodiment of the method, it is evaluated whether two criteria are fulfilled, wherein one of these criteria is an AF-dependent criterion, wherein the other criterion is an AF-independent criterion. This step is performed after the step of switching the operational mode of the implantable medical device from the atrial fibrillation mode to the atrial atrial-fibrillation optimized long-term mode or, alternatively, after the step of switching the operational mode of the implantable medical device from the atrial-fibrillation optimized long-term mode to the post-atrial fibrillation short-term mode. The AF-dependent criteria are the first and second criteria explained in the following. The AF-independent criteria are the third and fourth criteria explained in the following.

The first criterion is fulfilled if the atrial electric signal is no longer indicative for an atrial fibrillation. This criterion is an AF-dependent criterion that is independent on the operational mode of the implantable medical device.

The second criterion is fulfilled if the atrial electric signal has not been indicative for atrial fibrillation over a time period of at least 1 minute to 30 minutes, in particular over a time period lying in a range of from 1 minute to 30 minutes, in particular of from 1.5 minutes to 25 minutes, in particular 10 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes, preferably 10 minutes. Preferably, the second criterion is fulfilled if the atrial electric signal has not been indicative for atrial fibrillation over a time period of at least 15 minutes. This second criterion is also an AF-dependent criterion that is independent on the operational mode of the implantable medical device.

The third criterion is fulfilled if an atrial fibrillation mode termination criterion is fulfilled. Typically, such atrial fibrillation mode termination criterion is fulfilled if the atrial rate is not only lower than the atrial rate under atrial fibrillation, but also lower than the atrial rate under atrial tachycardia. However, other atrial fibrillation mode termination criteria are also generally possible. The atrial fibrillation mode termination criterion is an AF-independent criterion that is based on the operational mode of the implantable medical device.

The fourth criterion is fulfilled if an atrial fibrillation mode termination criterion has been fulfilled over a time period of at least 1 minute, in particular over a time period lying in a range of from 1 minute to 30 minutes, in particular of from 1.5 minutes to 25 minutes, in particular 10 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes, preferably 10 minutes.

If one the first and second criteria and if additionally one of the third and fourth criteria of the precedingly explained criteria are fulfilled, the operational mode of the implantable medical device is switched from either the atrial-fibrillation optimized long-term mode or from the confirmation phase to the regular mode. In this context, the regular mode exclusively allows an atrial controlled stimulation of the ventricle of the heart to be stimulated. At the same time, the regular mode comprises activated additional functions that comprise the activated additional functions of the atrial-fibrillation optimized long-term mode or the confirmation phase. The regular mode may comprise further activated additional functions with respect to the atrial-fibrillation optimized long-term mode or the confirmation phase. However, in some embodiments, the activated additional functions of the regular mode are identical to the activated additional functions of the atrial-fibrillation optimized long-term mode.

In an embodiment, the atrial fibrillation mode termination criterion is a detected atrial signal that is indicative for an atrial tachycardia but not yet indicative for an atrial fibrillation. Such an atrial electric signal indicative for an atrial tachycardia is a signal indicating an atrial rate higher than 80 bpm, in particular an atrial rate lying within a range of from 80 bpm to less than 200 bpm, in particular of from 90 bpm to 18 bpm, in particular of from 100 bpm to 170 bpm, in particular of from 110 bpm to 160 bpm, in particular 130 bpm, in particular of from 120 bpm to 140 bpm. Preferably, the atrial rate is higher than 180 bpm. As indicated above, an atrial rate being indicative for atrial fibrillation is typically an atrial rate of at least 180 bpm.

In an embodiment, the ventricular controlled stimulation of the ventricle of the heart to be stimulated is an R-wave synchronous stimulation. Such an R-wave synchronous stimulation can also be denoted as R synchronized stimulation. Thus, the stimulation is carried out using the R wave in the electric cardiac signal as reference point.

In an embodiment, the atrial controlled stimulation of the ventricle of the heart to be stimulated as a P-wave synchronous stimulation. Thus, in this case the P wave of the atrial cardiac signal is used as reference point for the ventricular stimulation. Such a P-wave synchronous stimulation can also be denoted as P synchronized stimulation.

In an embodiment, the regular mode employs i) an atrial and ventricular (dual) sensing, ii) an atrial and a ventricular pacing or a ventricular pacing only, and iii) a dual control mode enabling pacing or inhibiting of pacing in dependence on the sensed signals (i.e., a DDD or a VDD operational mode).

In an embodiment, the atrial fibrillation mode employs an atrial and ventricular (dual) sensing, an atrial and ventricular (dual) pacing and an inhibiting control mode enabling inhibition of pacing (i.e. a DDI operational mode). In another embodiment, the atrial fibrillation mode employs a ventricular sensing, a ventricular pacing and an inhibiting control mode enabling inhibition of pacing (i.e., a VVI operational mode).

In an embodiment, the atrial-fibrillation optimized long-term mode employs an atrial and ventricular (dual) sensing, an atrial and ventricular (dual) pacing and an inhibiting control mode enabling inhibition of pacing (i.e., a DDI mode). In another embodiment, the atrial-fibrillation optimized long-term mode employs ventricular sensing, ventricular pacing and an inhibiting control mode enabling inhibition of pacing (i.e., a VVI operational mode).

In an embodiment, the program causes the processor to reactivate all previously deactivated additional functions when switching the operational mode from the atrial fibrillation mode to the atrial-fibrillation optimized long-term mode. In doing so, the atrial-fibrillation optimized long-term mode offers the full functionality of the regular mode of the implantable medical device, but employs ventricular based timing for the pacing instead of an atrial based timing.

In an aspect, the present invention relates to a method for controlling an implantable medical device according to the preceding explanations. This method comprises the steps explained in the following.

In a method step, information is repeatedly provided on whether an atrial fibrillation (in particular in the right atrium) is detected in the heart to be stimulated. This information can be obtained, e.g., on the basis of an atrial electric signal.

If an atrial fibrillation has been detected, an operational mode of the implantable medical device is switched from a regular mode to an atrial fibrillation mode. In this context, the atrial fibrillation mode exclusively allows a ventricular controlled stimulation of the ventricle of the heart to be stimulated. At the same time, at least one additional function is deactivated. The at least one additional function uses information from the right atrium, when the right atrium is not in an AF state. In particular, the at least one additional function may be chosen from the group comprising, in particular consisting of, automatic pacing threshold monitoring, automatic sensing threshold monitoring, closed loop stimulation, beat-to-beat continuous capture control and automatic CRT-stimulation adaption.

In the further course of the method, it is evaluated whether a switching criterion is fulfilled. The switching criterion, in particular, may be at least one of the following four criteria. The first criterion is fulfilled if the atrial electric signal indicative for atrial fibrillation is detected over a time period of at least one hour, in particular over a time period lying within a range of from 15 minutes to 1 hour, from 1 hour to 12 hours, in particular of from 1.5 hours to 11 hours, in particular of from 2 hours to 10 hours, in particular of from 2.5 hours to 9 hours, in particular of from 3 hours to 8 hours, in particular of from 3.5 hours to 7 hours, in particular of from 4 hours to 6 hours, in particular of from 4.5 hours to 5 hours. In particular, the first criterion is fulfilled if the atrial electric signal indicative for atrial fibrillation is detected over a time period of at least 30 minutes. This first criterion is an AF-based criterion which is independent on the operational mode of the implantable medical device.

The second possible criterion is fulfilled if the atrial fibrillation mode is active over time period of at least one hour, in particular over a time period lying within a range of from 30 minutes to 1 hour, from 1 hour to 12 hours, in particular of from 1.5 hours to 11 hours, in particular of from 2 hours to 10 hours, in particular of from 2.5 hours to 9 hours, in particular of from 3 hours to 8 hours, in particular of from 3.5 hours to 7 hours, in particular of from 4 hours to 6 hours, in particular of from 4.5 hours to 5 hours. The second criterion is an AF-independent criterion that is based on the operational mode of the implantable medical device.

The third criterion is fulfilled if the atrial fibrillation mode is active over a cumulative time period of at least 2 hours within 24 hours, in particular over a cumulative time period lying in a range of from 2 hours to 12 hours, in particular of from 3 hours to 11 hours, in particular of from 4 hours to 10 hours, in particular of from 5 hours to 9 hours, in particular from 6 hours to 8 hours, within 24 hours. This third criterion is once again an AF-independent criterion that is based on the operational mode of the implantable medical device.

The fourth criterion is fulfilled if the atrial fibrillation mode has been activated at least 120 times within 24 hours, in particular at least 130 times within 24 hours, in particular at least 140 times within 24 hours, in particular at least 150 times within 24 hours, in particular at least 160 times within 24 hours, in particular at least 170 times within 24 hours, in particular at least 180 times within 24 hours, in particular at least 190 times within 24 hours, in particular at least 200 times within 24 hours.

If the switching criterion is fulfilled, this indicates that implantable medical device needs to deal with a long lasting atrial fibrillation. In such a case, the operational mode of the implantable medical device is switched from the atrial fibrillation mode to an atrial-fibrillation optimized long-term mode. This atrial-fibrillation optimized long-term mode exclusively allows a ventricular controlled stimulation of the ventricle of the heart to be stimulated. At the same time, at least one additional function, which, in particular, may be chosen from the group comprising, in particular consisting of, automatic pacing threshold monitoring, automatic sensing threshold monitoring, closed loop stimulation, beat-to-beat continuous capture control and automatic CRT-stimulation adaption, that was deactivated in the atrial fibrillation mode is reactivated. Thus, the atrial-fibrillation optimized long-term mode is intended to be applied over a longer period of time. For this purpose, functions that are deactivated in the atrial fibrillation mode but that are considered to have a particular positive impact for the patient are automatically reactivated so as to offer a patient a more appropriate therapy providing higher functionality than the limited atrial fibrillation mode. Due to the reactivated functions that had been deactivated in the atrial fibrillation mode, the atrial-fibrillation optimized long-term mode is particularly appropriate to provide a patient with the required pacing over an extended period of time during which the patient suffers from an atrial fibrillation.

In order to allow a termination of the atrial-fibrillation optimized long-term mode if it is considered to be no longer necessary, the method comprises, in an embodiment, the further step explained in the following. This step is performed after the step of switching the operational mode of the implantable medical device from the atrial fibrillation mode to the atrial-fibrillation optimized long-term mode.

It is determined if one of the two following criteria is fulfilled. The first criterion is fulfilled if an atrial fibrillation is no longer detected anymore. This criterion is an AF-dependent criterion that is independent on the operational mode of the implantable medical device.

The second criterion is fulfilled if an atrial fibrillation has not been detected over a time period of at least 1 minute, in particular over a time period lying in a range of from 1 minute to 30 minutes, in particular of from 1.5 minutes to 25 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes. This second criterion is also an AF-dependent criterion that is independent on the operational mode of the implantable medical device.

If one of the precedingly explained criteria is fulfilled, the operational mode of the implantable medical device is switched from the atrial-fibrillation optimized long-term mode to a confirmation phase. This post-fibrillation (i.e. post atrial fibrillation) short-term mode serves for an adequate pacing strategy during a period in which the patient does no longer suffer from atrial fibrillation but still has an atrial tachycardia, i.e. an atrial rate that is higher than the regular atrial rate. In the confirmation phase, an exclusively ventricular controlled stimulation of the ventricle of the heart to be stimulated is allowed. At the same time, the confirmation phase comprises activated additional functions that correspond to the activated additional functions of the atrial-fibrillation optimized long-term mode. The confirmation phase serves as transition state in which an evaluation is conducted to determine whether a switch of the implantable device to the regular mode, i.e. the atrial controlled stimulation mode, can be performed.

In an embodiment, the confirmation phase employs an atrial and a ventricular (dual) sensing, an atrial and a ventricular (dual) pacing and an inhibiting control mode enabling inhibiting of pacing in dependence on the sensed signals (i.e., it is operated under a DDI mode). In another embodiment, the confirmation phase employs a ventricular sensing, a ventricular pacing and an inhibiting control mode enabling inhibiting of pacing in dependence on the sensed signals (i.e., it is operated under a VVI mode).

In a further method step employed in an embodiment of the method, it is evaluated whether two criteria are fulfilled, wherein one of these criteria is an AF-dependent criterion, wherein the other criterion is an AF-independent criterion. This step is performed after the step of switching the operational mode of the implantable medical device from the atrial fibrillation mode to the atrial atrial-fibrillation optimized long-term mode or, alternatively, after the step of switching the operational mode of the implantable medical device from the atrial-fibrillation optimized long-term mode to the post-atrial fibrillation short-term mode. The AF-dependent criteria are the first and second criteria explained in the following. The AF-independent criteria are the third and fourth criteria explained in the following.

The first criterion is fulfilled if an atrial fibrillation is no longer detected anymore. This criterion is an AF-dependent criterion that is independent on the operational mode of the implantable medical device.

The second criterion is fulfilled if an atrial fibrillation has not been detected over a time period of at least 1 minute, in particular over a time period lying in a range of from 1 minute to 30 minutes, in particular of from 1.5 minutes to 25 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes. This second criterion is also an AF-dependent criterion that is independent on the operational mode of the implantable medical device.

The third criterion is fulfilled if an atrial fibrillation mode termination criterion is fulfilled. Typically, such atrial fibrillation mode termination criterion is fulfilled if the atrial rate is not only lower than the atrial rate under atrial fibrillation, but also lower than the atrial rate under atrial tachycardia. However, other atrial fibrillation mode termination criteria are also generally possible. The atrial fibrillation mode termination criterion is an AF-independent criterion that is based on the operational mode of the implantable medical device.

The fourth criterion is fulfilled if an atrial fibrillation mode termination criterion has been fulfilled over a time period of at least 1 minute, in particular over a time period lying in a range of from 1 minute to 30 minutes, in particular of from 1.5 minutes to 25 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes.

If one the first and second criteria and if additionally one of the third and fourth criteria of the precedingly explained criteria are fulfilled, the operational mode of the implantable medical device is switched from either the atrial-fibrillation optimized long-term mode or from the confirmation phase to the regular mode. In this context, the regular mode exclusively allows an atrial controlled stimulation of the ventricle of the heart to be stimulated. At the same time, the regular mode comprises activated additional functions that comprise the activated additional functions of the atrial-fibrillation optimized long-term mode or the confirmation phase. The regular mode may comprise further activated additional functions with respect to the atrial-fibrillation optimized long-term mode or the confirmation phase. However, in some embodiments, the activated additional functions of the regular mode are identical to the activated additional functions of the atrial-fibrillation optimized long-term mode or the confirmation phase.

In an aspect, the present invention relates to computer program product comprising computer-readable code that causes the processor to perform the steps explained in the following when executed on the processor.

In a method step, an atrial electric signal is repeatedly detected with a first detection unit of an implantable medical device.

Based on the detected atrial signal, it is repeatedly determined whether an atrial fibrillation is present. Such determination can be easily done, in an embodiment, on the basis of the rate of the detected atrial signal. If the rate exceeds a threshold lying in a range of from 180 beats per minute (bpm) to 300 bpm, in particular of from 190 bpm to 290 bpm, in particular of from 200 bpm to 280 bpm, in particular of from 210 bpm to 270 bpm, in particular of from 220 bpm to 260 bpm, in particular of from 230 bpm to 250 bpm, the detected atrial electric signal is considered to be indicative for an atrial fibrillation.

If the atrial electric signal has been determined to be indicative for an atrial fibrillation, an operational mode of the implantable medical device is switched from a regular mode to an atrial fibrillation mode. In this context, the atrial fibrillation mode exclusively allows a ventricular controlled stimulation of the ventricle of the heart to be stimulated. At the same time, at least one additional function is deactivated. The at least one additional function uses information from the right atrium, when the right atrium is not in an AF state. The at least one additional function, in particular, may be chosen from the group comprising, in particular consisting of, automatic pacing threshold monitoring, automatic sensing threshold monitoring, closed loop stimulation, beat-to-beat continuous capture control and automatic CRT-stimulation adaption.

In the further course of the method, it is evaluated whether a switching criterion is fulfilled. In particular, the switching criterion may be one of the following four criteria. The first criterion is fulfilled if the atrial electric signal indicative for atrial fibrillation is detected over a time period of at least one hour, in particular over a time period lying within a range of from 30 minutes to 1 hour, from 1 hour to 12 hours, in particular of from 1.5 hours to 11 hours, in particular of from 2 hours to 10 hours, in particular of from 2.5 hours to 9 hours, in particular of from 3 hours to 8 hours, in particular of from 3.5 hours to 7 hours, in particular of from 4 hours to 6 hours, in particular of from 4.5 hours to 5 hours. This first criterion is an AF-based criterion which is independent on the operational mode of the implantable medical device.

The second possible criterion is fulfilled if the atrial fibrillation mode is active over time period of at least one hour, in particular over a time period lying within a range of from 30 minutes to 1 hour, from 1 hour to 12 hours, in particular of from 1.5 hours to 11 hours, in particular of from 2 hours to 10 hours, in particular of from 2.5 hours to 9 hours, in particular of from 3 hours to 8 hours, in particular of from 3.5 hours to 7 hours, in particular of from 4 hours to 6 hours, in particular of from 4.5 hours to 5 hours. The second criterion is an AF-independent criterion that is based on the operational mode of the implantable medical device.

The third criterion is fulfilled if the atrial fibrillation mode is active over a cumulative time period of at least 2 hours within 24 hours, in particular over a cumulative time period lying in a range of from 2 hours to 12 hours, in particular of from 3 hours to 11 hours, in particular of from 4 hours to 10 hours, in particular of from 5 hours to 9 hours, in particular from 6 hours to 8 hours, within 24 hours. This third criterion is once again an AF-independent criterion that is based on the operational mode of the implantable medical device.

The fourth criterion is fulfilled if the atrial fibrillation mode has been activated at least 120 times within 24 hours, in particular at least 130 times within 24 hours, in particular at least 140 times within 24 hours, in particular at least 150 times within 24 hours, in particular at least 160 times within 24 hours, in particular at least 170 times within 24 hours, in particular at least 180 times within 24 hours, in particular at least 190 times within 24 hours, in particular at least 200 times within 24 hours.

If the precedingly explained switching criterion is fulfilled, this indicates that a patient suffers from a long lasting atrial fibrillation. In such a case, the operational mode of the implantable medical device is switched from the atrial fibrillation mode to an atrial-fibrillation optimized long-term mode. This atrial-fibrillation optimized long-term mode exclusively allows a ventricular controlled stimulation of the ventricle of the heart to be stimulated. At the same time, the at least one additional function, which, in particular, may be chosen from the group comprising, in particular consisting of, automatic pacing threshold monitoring, automatic sensing threshold monitoring, closed loop stimulation, beat-to-beat continuous capture control and automatic CRT-stimulation adaption, that was deactivated in the atrial fibrillation mode is reactivated. Thus, the atrial-fibrillation optimized long-term mode is intended to be applied over a longer period of time. For this purpose, functions that are deactivated in the atrial fibrillation mode but that are considered to have a particular positive impact for the patient are automatically reactivated so as to offer the patient a more appropriate therapy providing higher functionality than the limited atrial fibrillation mode.

The computer program comprises, in an embodiment, the further step explained in the following. This step is performed after the step of switching the operational mode of the implantable medical device from the atrial fibrillation mode to the atrial-fibrillation optimized long-term mode.

It is determined if one of the two following criteria is fulfilled. The first criterion is fulfilled if the atrial electric signal is no longer indicative for an atrial fibrillation. This criterion is an AF-dependent criterion that is independent on the operational mode of the implantable medical device.

The second criterion is fulfilled if the atrial electric signal has not been indicative for atrial fibrillation over a time period of at least 1 minute, in particular over a time period lying in a range of from 1 minute to 30 minutes, in particular of from 1.5 minutes to 25 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes. This second criterion is also an AF-dependent criterion that is independent on the operational mode of the implantable medical device.

If one of the precedingly explained criteria is fulfilled, the operational mode of the implantable medical device is switched from the atrial-fibrillation optimized long-term mode to a confirmation phase. This post-fibrillation (i.e. post atrial fibrillation) short-term mode serves for an adequate pacing strategy during a period in which the patient does no longer suffer from atrial fibrillation but still has an atrial tachycardia, i.e. an atrial rate that is higher than the regular atrial rate. In the confirmation phase, an exclusively ventricular controlled stimulation of the ventricle of the heart to be stimulated is allowed. The confirmation phase serves as transition state in which an evaluation is conducted to determine whether a switch of the implantable device to the regular mode, i.e. the atrial controlled stimulation mode, can be performed.

In a further step employed in an embodiment of the computer program, it is evaluated whether two criteria are fulfilled, wherein one of these criteria is an AF-dependent criterion, wherein the other criterion is an AF-independent criterion. This step is performed after the step of switching the operational mode of the implantable medical device from the atrial fibrillation mode to the atrial atrial-fibrillation optimized long-term mode or, alternatively, after the step of switching the operational mode of the implantable medical device from the atrial-fibrillation optimized long-term mode to the post-atrial fibrillation short-term mode. The AF-dependent criteria are the first and second criteria explained in the following. The AF-independent criteria are the third and fourth criteria explained in the following.

The first criterion is fulfilled if the atrial electric signal is no longer indicative for an atrial fibrillation. This criterion is an AF-dependent criterion that is independent on the operational mode of the implantable medical device.

The second criterion is fulfilled if the atrial electric signal has not been indicative for atrial fibrillation over a time period of at least 1 minute, in particular over a time period lying in a range of from 1 minute to 30 minutes, in particular of from 1.5 minutes to 25 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes. This second criterion is also an AF-dependent criterion that is independent on the operational mode of the implantable medical device.

The third criterion is fulfilled if an atrial fibrillation mode termination criterion is fulfilled. Typically, such atrial fibrillation mode termination criterion is fulfilled if the atrial rate is not only lower than the atrial rate under atrial fibrillation, but also lower than the atrial rate under atrial tachycardia. However, other atrial fibrillation mode termination criteria are also generally possible. The atrial fibrillation mode termination criterion is an AF-independent criterion that is based on the operational mode of the implantable medical device.

The fourth criterion is fulfilled if an atrial fibrillation mode termination criterion has been fulfilled over a time period of at least 1 minute, in particular over a time period lying in a range of from 1 minute to 30 minutes, in particular of from 1.5 minutes to 25 minutes, in particular from 2 minutes to 20 minutes, in particular of from 3 minutes to 17.5 minutes, in particular of from 3.5 minutes to 15 minutes, in particular of from 4 minutes to 14 minutes, in particular from 5 minutes to 13 minutes, in particular of from 6 minutes to 12 minutes, in particular from 7 minutes to 11 minutes, in particular from 8 minutes to 10 minutes.

If one the first and second criteria and if additionally one of the third and fourth criteria of the precedingly explained criteria are fulfilled, the operational mode of the implantable medical device is switched from either the atrial-fibrillation optimized long-term mode or from the confirmation phase to the regular mode. In this context, the regular mode exclusively allows an atrial controlled stimulation of the ventricle of the heart to be stimulated. At the same time, the regular mode comprises activated additional functions that comprise the activated additional functions of the atrial-fibrillation optimized long-term mode. The regular mode may comprise further activated additional functions with respect to the atrial-fibrillation optimized long-term mode. However, in some embodiments, the activated additional functions of the regular mode are identical to the activated additional functions of the atrial-fibrillation optimized long-term mode.

In an aspect, the present invention relates to a method of treatment of a human or animal patient in need of such treatment with an implantable medical device for stimulating a human or animal heart, in particular with an implantable medical device according to the preceding explanations. Such an implantable medical device comprises a processor, a memory unit, a stimulation unit, a first detection unit, and a second detection unit. The stimulation unit serves for stimulating a human or animal heart. The first detection unit serves for detecting an electric signal in an atrium of this heart. The second detection unit serves for detecting an electric signal in a ventricle of the heart.

The method comprises the following steps: a) repeatedly detecting, with the first detection unit, an atrial electric signal; b) repeatedly determining whether the atrial electric signal is indicative for an atrial fibrillation; c) if the atrial electric signal is indicative for an atrial fibrillation, switching an operational mode of the implantable medical device from a regular mode to a fibrillation mode, wherein the fibrillation mode exclusively allows a ventricular controlled stimulation of a ventricle of the heart to be stimulated, wherein at least one additional function that uses information from the right atrium, when the right atrium is not in an AF state is deactivated; d) switching the operational mode of the implantable medical device from the fibrillation mode to an atrial-fibrillation optimized long-term mode, if a switching criterion is fulfilled;

wherein the atrial-fibrillation optimized long-term mode exclusively allows a ventricular controlled stimulation of the ventricle of the heart to be stimulated, wherein the at least one additional function that was deactivated in the fibrillation mode is reactivated.

In particular, the at least one additional function may be chosen from the group comprising automatic pacing threshold monitoring, automatic sensing threshold monitoring, closed loop stimulation, beat-to-beat continuous capture control and automatic CRT-stimulation adaption.

In particular, the switching criterion may be at least one of the following criteria: i) the atrial electric signal indicative for an atrial fibrillation is detected over a time period of at least 15 minutes to 1 hour, ii) the fibrillation mode is active over a time period of at least 15 minutes to 1 hour, iii) the fibrillation mode is active over a cumulative time period of at least 2 hours within 24 hours, iv) the fibrillation mode (222) was activated at least 120 (preferably 200) times within 24 hours.

In addition, cardiac pacing is performed on the basis of the operational mode to which the operation of the implantable medical device has been switched by the executed method.

All embodiments of the implantable medical device can be combined in any desired way and can be transferred either individually or in any arbitrary combination to the described methods and the described computer program product. Likewise, all embodiments of the described methods can be combined in any desired way and can be transferred either individually or in any arbitrary combination to the respective other method, to the implantable medical device and to the computer program product. Finally, all embodiments described with respect to the computer program product can be combined in any desired way and can be transferred either individually or in any arbitrary combination to the described implantable medical device or to the described methods.

### BRIEF DESCRIPTION OF THE DRAWINGS

Further details of aspects of the present invention will be explained with respect to exemplary embodiments and accompanying Figures. In the Figures:

FIG. 1 shows a schematic depiction of a prior art method for switching the operational mode of an implantable medical device in response to a detected atrial fibrillation;

FIG. 2 shows a first embodiment of a method for switching the operational mode of an implantable medical device in response to a detected atrial fibrillation and to automatically adopt an atrial-fibrillation optimized long-term mode; and

FIG. 3 shows a second embodiment of a method for switching the operational mode of an implantable medical device in response to a detected atrial fibrillation and to automatically adopt an atrial-fibrillation optimized long-term mode.

### DETAILED DESCRIPTION

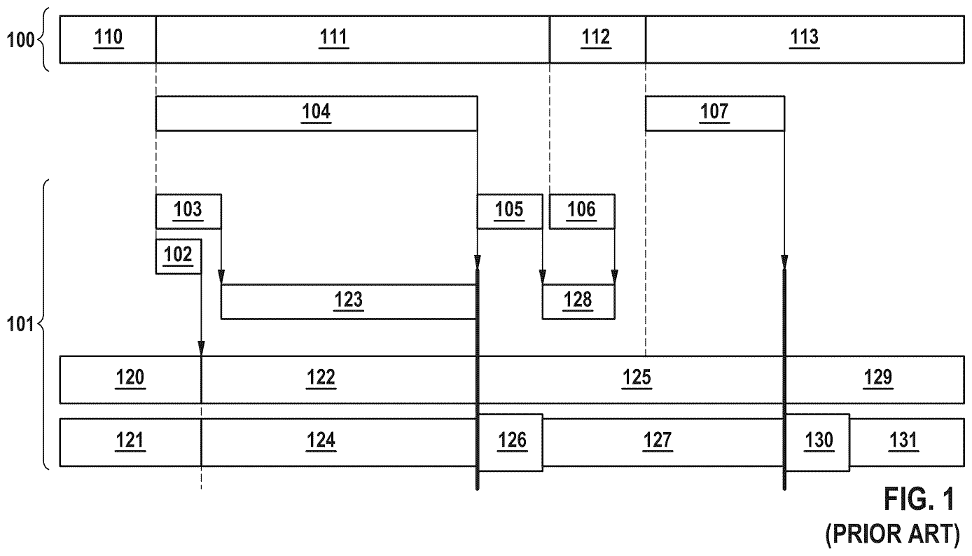

FIG. 1 shows a schematic depiction of a prior art method for switching the operational mode of an implantable medical device in response to a detected atrial fibrillation.

In an upper portion 100 of FIG. 1, the cardiac state of a patient as indicated. In a lower portion 101 of FIG. 1, the operational mode of a cardiac pacemaker (i.e., an implantable medical device for stimulating the heart of the patient) is indicated. The heart of the patient initially shows a normal cardiac rate 110. Afterwards, the cardiac state of the heart of the patient changes to an atrial fibrillation 111, followed by an atrial tachycardia 112 (high atrial rate yet lower than the rate under atrial fibrillation), finally followed once again by a normal atrial rate 113. The cardiac pacemaker is active in a regular operational mode 120 that works in a P synchronous manner, i.e. under atrial control. All algorithms 121 are allowed and active.

After a mode switch detection time 102, the cardiac pacemaker switches to a mode switch mode 122. In this mode switch mode 122, the cardiac pacemaker is operated in R synchronous manner, i.e. under ventricular control. After an atrial fibrillation/atrial tachycardia (AF/AT) detection time 103, a first atrial fibrillation episode 123 of the patient is detected by the cardiac pacemaker.

During the mode switch mode 122, a plurality of algorithms 124 is suspended or stopped. Thus, the mode switch mode 122 only offers limited functionality on the basis of a limited number of available algorithms 124. After a first time period 104 until a first follow-up, a physician can manually change the operational mode of the cardiac pacemaker from the mode switch mode 122 to an R synchronous normal mode 125. At the beginning of this R synchronous normal mode 125, a re-initialization 126 of the previously deactivated algorithms in R synchronous operational mode takes place. Afterwards, all these algorithms 127 are allowed and available in R synchronous mode. After a second AF/AT detection time 105, the cardiac pacemaker detects a second atrial fibrillation episode 128 of the patient. This second atrial fibrillation episode 128 of the patient is terminated after an AF/AT termination time 106 starting at the beginning of the atrial tachycardia 112.

The R synchronous normal mode 125 is active until a second time period 107 lasting up to a further follow-up has been passed. Then, a physician can change the R synchronous normal mode 125 to a P synchronous normal mode 129 that corresponds to the initial P synchronous regular (normal) mode 120. At the beginning of the P synchronous normal mode 129, a re-initialization 130 of a plurality of algorithms in a P synchronous mode takes place. Afterwards, all algorithms 131 are allowed and available in P synchronous mode.

Thus, the change of the mode switch mode 122 to an R synchronous normal mode 125 is dependent on the first time period 104 to a follow-up and on the manual interaction of a physician. Furthermore, the R synchronous normal mode continues to be active even after termination of the atrial fibrillation 111 or a subsequent atrial tachycardia 112, i.e., also during a period of normal atrial rate 113 until a second time period 107 lasting up to the second follow-up has been passed. Thus, the operational mode of the cardiac pacemaker does not reflect at all the physiologic conditions in the heart to be stimulated.

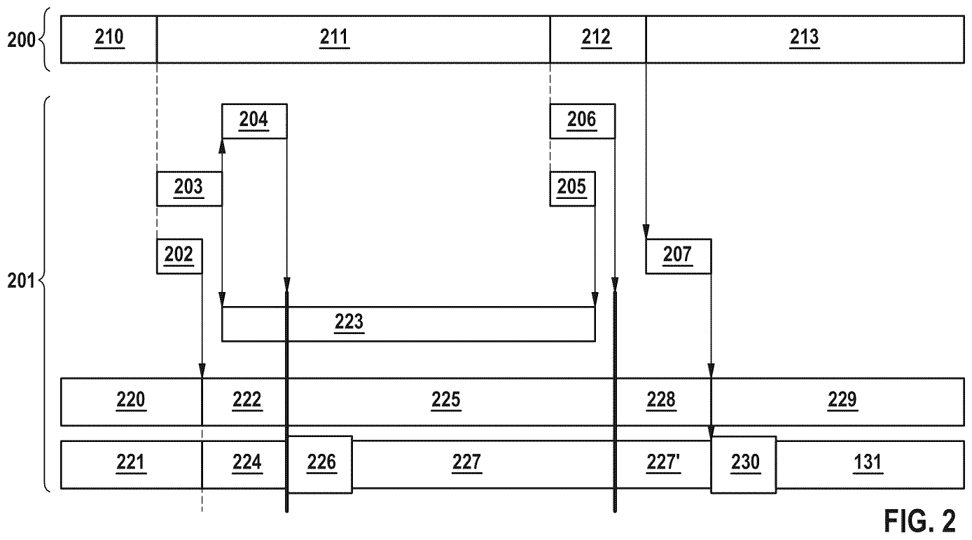

FIG. 2 shows a first embodiment of a method for switching the operational mode of an implantable medical device in response to a detected atrial fibrillation and automatically adopting an atrial-fibrillation optimized long-term mode.

Like in FIG. 1, a condition 200 of a heart to be treated is indicated in an upper portion of FIG. 2. The sate 201 of a cardiac pacemaker used for treating this heart is indicated in the lower portion of FIG. 2.

The condition 200 of the heart to be treated in FIG. 2 is similar to that of FIG. 1. Thus, initially, a normal atrial rate 210 is present that is followed by an atrial fibrillation 211 which, in turn, is followed by an atrial tachycardia 212 with an atrial rate being lower than in case of atrial fibrillation but still higher than in case of a normal atrial rate. Finally, the heart returns to a normal atrial rate 213.

Initially, the cardiac pacemaker used to treat the heart is operated in its normal mode 220. This normal mode 220 is a P synchronous mode, i.e., it employs an atrial control. During the normal mode 220, all algorithms 221 are allowed and activated.

After a mode switch detection time 202, the operational mode of the cardiac pacemaker is switched to an atrial fibrillation mode 222. This atrial fibrillation mode 222 is an R synchronous operational mode, i.e., an operational mode under ventricular control. In the atrial fibrillation mode 221, a plurality of algorithms 224 is suspended and stopped. Thus, the fibrillation mode 221 only offers a limited functionality.

After a first AF/AT detection time 203, an atrial fibrillation episode 223 is detected. After a second AF/AT detection time 204, this atrial fibrillation episode is classified as long-term atrial fibrillation. Consequently, the operational mode of the cardiac pacemaker is switched from the fibrillation mode 222 to an atrial-fibrillation optimized long-term mode 225. This atrial-fibrillation optimized long-term mode is an R synchronous mode, i.e., a mode under ventricular control. A re-initialization 226 of the previously deactivated algorithms in R synchronous mode is done. Consequently, all algorithms 227 are allowed and available in R synchronous manner during the atrial-fibrillation optimized long-term mode 225.

After the end of a first AF/AT termination time 205, the atrial fibrillation episode 223 is declared to be terminated. After the end of a second AF/AT termination time 206 that is longer than the first AF/AT termination time 205, a termination of a long-term AF/AT is determined. Consequently, the operational mode of the cardiac pacemaker is switched from the atrial-fibrillation optimized long-term mode 225 to a confirmation phase 228. This confirmation phase 228 is-like the atrial-fibrillation optimized long-term mode 225—an R synchronous mode, i.e., an operational mode under ventricular control. Consequently, the algorithms 227 and 227′ are still operated in R synchronous mode. Alternatively, a plurality of algorithms 227′ is suspended and stopped after the end of second AF/AT termination time 206. Introducing the short-term R-wave synchronized mode 227′ is beneficial regarding reinitialization and activation of the functions 227 in the P-wave synchronized long term mode. After the termination of a mode switch terminating time 207, the operational mode of the cardiac pacemaker is switched from the confirmation phase 228 to a normal mode 229. This normal mode 229 is a P synchronous mode, i.e., a mode under atrial control. It generally corresponds to the initial normal mode 220. Consequently, a re-initialization 230 of the available algorithms in P synchronous mode is carried out so that afterwards all algorithms 231 are available and fully operational in P synchronous mode.

The automatic switching of the operational mode of the cardiac pacemaker after having detected a long persisting atrial fibrillation to the atrial-fibrillation optimized long-term mode 225 and the automatic back switch to a confirmation phase 228 and finally to a regular mode 229 under atrial control offers a fully automatic operation of the cardiac pacemaker that does not require human interaction in a follow-up as this is the case in prior art pacemakers. The pacemaker operated under the method explained with respect to FIG. 2 adjusts itself to the physiological needs of the heart to be stimulated and thus offers a highly physiologic pacing strategy that enables a particular advantageous pacing of the heart to be stimulated.

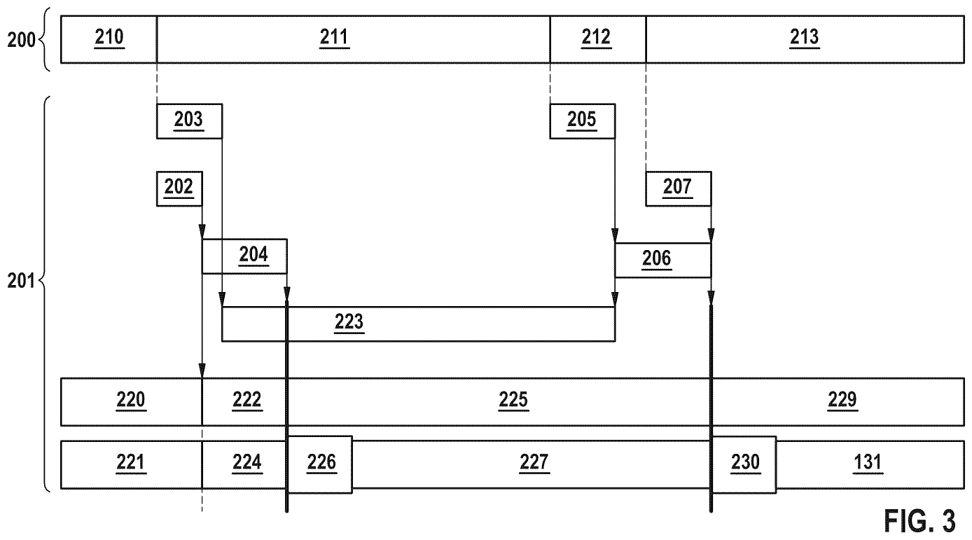

FIG. 3 shows a second embodiment of a method for switching the operational mode of an implantable medical device in response to detected atrial fibrillation and automatically adopting an atrial-fibrillation optimized long-term mode. The method explained with respect to FIG. 3 also considers a mode switch criterion for terminating the atrial-fibrillation optimized long-term mode.

This embodiment generally resembles the embodiment shown in FIG. 2. Therefore, the same numeral references are used for the same cardiac states, operational modes, and further elements of the method, such as time periods. Referring to the explanations given with respect to FIG. 2, in the following only the differences between the embodiment shown in FIG. 3 and the embodiment shown in FIG. 2 will be explained.

In the embodiment shown in FIG. 3, the second AF/AT detection time 204 already starts at the end of the mode switch detection time 202 and not—as in case of the embodiment of FIG. 2—at the end of the first AF/AT detection time 203. Consequently, the atrial-fibrillation optimized long-term mode 225 starts in the embodiment of FIG. 3 earlier than in the embodiment of FIG. 2. While the embodiment of FIG. 2 offers a confirmation phase 228, no such mode is present in the embodiment of FIG. 3. Rather, the second AF/AT termination time 206 only starts at the end of the first AF/AT termination time 205. Only at a time point at which both the second AF/AT termination time 206 and the mode switch termination time 207 are terminated, the operational mode of the cardiac pacemaker is switched from the atrial-fibrillation optimized long-term mode 225 to the P synchronous regular mode 229 after the heart has been returned to its normal atrial rate 213. In this regular mode 229, all algorithms 231 are allowed and available and operated in P synchronous manner.

The small variations between the embodiment shown in FIG. 2 and the embodiment shown in FIG. 3 do not alter the general superiority of the presently described method with respect to prior art methods. Rather, also the embodiment shown in FIG. 3 offers a highly physiologic response of the cardiac pacemaker to the cardiac state of the heart to be treated. The method serves for an automatic switch of the operational mode of the cardiac pacemaker to an atrial-fibrillation optimized long-term mode during which a high number of functions/algorithms is available and activated so that an optimized pacing of the heart to be stimulated is possible even in case of a longer persisting atrial fibrillation. Furthermore, the cardiac pacemaker operated under the method explained in FIG. 3 automatically switches to its regular mode 229 once the atrial fibrillation (and a subsequent atrial tachycardia) has been terminated and the heart has been returned to its normal atrial rate 213.

The Present Invention Also Includes the Following Aspects:
1. Implantable medical device for stimulating a human or animal heart, comprising a processor, a memory unit, a stimulation unit configured to stimulate a human or animal heart, a first detection unit configured to detect an electric signal in an atrium of the same heart, and a second detection unit configured to detect an electric signal in a ventricle of the same heart,
   characterized
   in that the memory unit comprises a computer-readable program that causes the processor to perform the following steps when executed on the processor:
   a) repeatedly detecting, with the first detection unit, an atrial electric signal;
   b) repeatedly determining whether the atrial electric signal is indicative for an atrial fibrillation (211);
   c) if the atrial electric signal is indicative for an atrial fibrillation (211), switching an operational mode of the implantable medical device from a regular mode (220) to a fibrillation mode (222), wherein the fibrillation mode (222) exclusively allows a ventricular controlled stimulation of a right ventricle of the heart to be stimulated, wherein at least one additional function chosen from the group comprising automatic pacing threshold monitoring, automatic sensing threshold monitoring, closed loop stimulation, beat-to-beat continuous capture control and automatic CRT-stimulation adaption is deactivated;
   and
   switching the operational mode of the implantable medical device from the fibrillation mode (222) to an atrial-fibrillation optimized long-term mode (225),
   d) if at least one the following criteria is fulfilled:
      i) the atrial electric signal indicative for an atrial fibrillation (211) is detected over a time period of at least 15 minutes,
      ii) the fibrillation mode (222) is active over a time period of at least 15 minutes,
      iii) the fibrillation mode (222) is active over a cumulative time period of at least 2 hours within 24 hours,
      iv) the fibrillation mode (222) was activated at least 120 times within 24 hours,
wherein the atrial-fibrillation optimized long-term mode (225) exclusively allows a ventricular controlled stimulation of the right ventricle of the heart to be stimulated, wherein at least one additional function chosen from the group comprising automatic pacing threshold monitoring, automatic sensing threshold monitoring, closed loop stimulation, beat-to-beat continuous capture control and automatic CRT-stimulation adaption that was deactivated in the fibrillation mode (222) is reactivated (226).
2. Implantable medical device according to aspect 1, characterized in that the program causes the processor to perform the following step after step d): switching the operational mode of the implantable medical device from the atrial-fibrillation optimized long-term mode (225) to a confirmation phase confirmation phase (228), if at least one the following criteria is fulfilled:
   i) the atrial electric signal is no longer indicative for an atrial fibrillation (211),
   ii) the atrial electric signal has not been indicative for an atrial fibrillation (211) over a time period of at least 1 minute to 30 minutes,
wherein the confirmation phase (228) exclusively allows a ventricular controlled stimulation of the right ventricle of the heart to be stimulated, wherein the confirmation phase (228) comprises activated additional functions that correspond to the activated additional functions of the atrial-fibrillation optimized long-term mode (225), and
wherein the program causes the processor to perform a evaluate if a switch of the implantable device to the regular mode (220) can be performed.
3. Implantable medical device according to aspect 2, characterized in that the evaluate comprises evaluation if among the last Y number of heart cycles, an X number of heart cycles occurred with an atrial heart rate below a threshold rate.
4. Implantable medical device according to any of the preceding aspects, characterized in that the program causes the processor to perform the following step after step d): switching the operational mode of the implantable medical device from either the atrial-fibrillation optimized long-term mode (225) or the confirmation phase (228) to a regular mode (229), if at least one the following criteria i) and ii) and additionally at least one the following criteria iii) and iv) is fulfilled:
   i) the atrial electric signal is no longer indicative for an atrial fibrillation (211),
   ii) the atrial electric signal has not been indicative for an atrial fibrillation (211) over a time period of at least 1 minute,
   iii) a fibrillation mode termination criterion is fulfilled,
   iv) a fibrillation mode termination criterion has been fulfilled over a time period of at least 1 minute,
wherein the regular mode (229) exclusively allows an atrial controlled stimulation of the right ventricle of the heart to be stimulated, wherein the regular mode (229) comprises activated additional functions that comprise the activated additional functions of the atrial-fibrillation optimized long-term mode (225).
5. Implantable medical device according to aspect 4, characterized in that the fibrillation mode termination criterion is a detected atrial electric signal that is indicative for an atrial rate higher than 80 bpm but lower than an atrial rate under atrial fibrillation (211).
6. Implantable medical device according to any of the preceding aspects, characterized in that the ventricular controlled stimulation of the right ventricle of the heart to be stimulated is an R-wave synchronous stimulation.
7. Implantable medical device according to any of the preceding aspects, characterized in that the atrial controlled stimulation of the right ventricle of the heart to be stimulated is a P-wave synchronous stimulation.
8. Implantable medical device according to any of the preceding aspects, characterized in that the regular mode (229) employs an atrial and a ventricular sensing, an atrial and/or a ventricular pacing and a dual control mode enabling pacing or inhibition of pacing in dependence on the sensed signals.
9. Implantable medical device according to any of the preceding aspects, characterized in that the fibrillation mode (222) employs i) an atrial and a ventricular sensing, an atrial and a ventricular pacing and an inhibiting control mode enabling inhibition of pacing or ii) a ventricular sensing, a ventricular pacing and an inhibiting control mode enabling inhibition of pacing.
10. Implantable medical device according to any of the preceding aspects, characterized in that the atrial-fibrillation optimized long-term mode (225) employs i) an atrial and a ventricular sensing, an atrial and a ventricular pacing and an inhibiting control mode enabling inhibition of pacing or ii) a ventricular sensing, a ventricular pacing and an inhibiting control mode enabling inhibition of pacing.
11. Implantable medical device according to any of the preceding aspects, characterized in that the program causes the processor to reactivate all previously deactivated additional functions when switching the operational mode from the fibrillation mode (222) to the atrial-fibrillation optimized long-term mode (225).
12. Method for controlling an implantable medical device according to any of the preceding aspects, comprising the following steps:
   characterized
   a) repeatedly providing information on whether an atrial fibrillation (211) is detected in a heart to be stimulated;
   b) if an atrial fibrillation (211) has been detected, switching an operational mode of the implantable medical device from an initial regular mode (220) to a fibrillation mode (222), wherein the fibrillation mode (222) exclusively allows a ventricular controlled stimulation of a right ventricle of the heart to be stimulated, wherein at least one additional function chosen from the group comprising automatic pacing threshold monitoring, automatic sensing threshold monitoring, closed loop stimulation, beat-to-beat continuous capture control and automatic CRT-stimulation adaption is deactivated; and
   switching the operational mode of the implantable medical device from the fibrillation mode (222) to an atrial-fibrillation optimized long-term mode (225),
   c) if at least one the following criteria is fulfilled:
      i) the atrial fibrillation (211) is detected over a time period of at least 15 minutes,
      ii) the fibrillation mode (222) is active over a time period of at least 1 hour,
      iii) the fibrillation mode (222) is active over a cumulative time period of at least 2 hours within 24 hours,
      iv) the fibrillation mode (222) was activated at least 120 (preferably 200) times within 24 hours,
wherein the atrial-fibrillation optimized long-term mode (225) exclusively allows a ventricular controlled stimulation of the right ventricle of the heart to be stimulated, wherein at least one additional function chosen from the group comprising automatic pacing threshold monitoring, automatic sensing threshold monitoring, closed loop stimulation, beat-to-beat continuous capture control and automatic CRT-stimulation adaption that was deactivated in the fibrillation mode (222) is reactivated (226).
13. Method according to aspect 12, characterized in that the method comprises the following further step performed after step c): switching the operational mode of the implantable medical device from the atrial-fibrillation optimized long-term mode (225) to a confirmation phase (228), if at least one the following criteria is fulfilled:
   i) an atrial fibrillation (211) is no longer detected,
   ii) an atrial fibrillation (211) has not been detected over a time period of at least 1 minute,
wherein the confirmation phase (228) exclusively allows a ventricular controlled stimulation of the right ventricle of the heart to be stimulated, wherein the confirmation phase (228) comprises activated additional functions that correspond to the activated additional functions of the atrial-fibrillation optimized long-term mode (225),
evaluating in the confirmation phase (228) if a switch of the implantable device to the regular mode (220) can be performed.
14. Method according to aspect 12 or 13, characterized in that the method comprises the following further step performed after step c): switching the operational mode of the implantable medical device from either the atrial-fibrillation optimized long-term mode (225) or the confirmation phase (228) to a regular mode (229), if at least one the following criteria i) and ii) and additionally at least one the following criteria iii) and iv) is fulfilled:
   i) an atrial fibrillation (211) is no longer detected,
   ii) an atrial fibrillation (211) has not been detected over a time period of at least 1 minute,
   iii) a fibrillation mode termination criterion is fulfilled,
   iv) a fibrillation mode termination criterion has been fulfilled over a time period of at least 1 minute,
wherein the regular mode (229) exclusively allows an atrial controlled stimulation of the right ventricle of the heart to be stimulated, wherein the regular mode (229) comprises activated additional functions that comprise the activated additional functions of the atrial-fibrillation optimized long-term mode (225) or of the confirmation phase (228).
15. Computer program product comprising computer-readable code that causes a processor to perform the following steps when executed on the processor:
   ii) repeatedly detecting, with a first detection unit of an implantable medical device for stimulating a human or animal heart, an atrial electric signal;
   b) repeatedly determining whether the atrial electric signal is indicative for an atrial fibrillation (211);
   c) if the atrial electric signal is indicative for an atrial fibrillation (211), switching an operational mode of the implantable medical device from an initial regular mode (220) to a fibrillation mode (222), wherein the fibrillation mode (222) exclusively allows a ventricular controlled stimulation of a right ventricle of the heart to be stimulated, wherein at least one additional function chosen from the group comprising automatic pacing threshold monitoring, automatic sensing threshold monitoring, closed loop stimulation, beat-to-beat continuous capture control and automatic CRT-stimulation adaption, is deactivated;
      switching the operational mode of the implantable medical device from the fibrillation mode (222) to an atrial-fibrillation optimized long-term mode (225),
   d) if at least one the following criteria is fulfilled:
      i) the atrial electric signal indicative for an atrial fibrillation (211) is detected over a time period of at least 1 hour,
      ii) the fibrillation mode (222) is active over a time period of at least 1 hour,
      iii) the fibrillation mode (222) is active over a cumulative time period of at least 2 hours within 24 hours,
      iv) the fibrillation mode (222) was activated at least 120 (preferably 200) times within 24 hours,
      wherein the atrial-fibrillation optimized long-term mode (225) exclusively allows a ventricular controlled stimulation of the right ventricle of the heart to be stimulated, wherein at least one additional function chosen from the group comprising automatic pacing threshold monitoring, automatic sensing threshold monitoring, closed loop stimulation, beat-to-beat continuous capture control and automatic CRT-stimulation adaption, that was deactivated in the fibrillation mode (222) is reactivated (226);
   e) optionally, if at least one the following criteria is fulfilled:
      i) the atrial electric signal is no longer indicative for an atrial fibrillation (211),
      ii) the atrial electric signal has not been indicative for an atrial fibrillation (211) over a time period of at least 1 minute,
      switching the operational mode of the implantable medical device from the atrial-fibrillation optimized long-term mode (225) to a confirmation phase (228), wherein the confirmation phase (228) exclusively allows a ventricular controlled stimulation of the right ventricle of the heart to be stimulated, wherein the confirmation phase (228) comprises activated additional functions that correspond to the activated additional functions of the atrial-fibrillation optimized long-term mode (225),
      evaluating in the confirmation phase (228) if a switch of the implantable device to the regular mode (220) can be performed;
   f) optionally, if at least one the following criteria i) and ii) and additionally at least one the following criteria iii) and iv) are fulfilled:
      i) the atrial electric signal is no longer indicative for an atrial fibrillation (211),
      ii) the atrial electric signal has not been indicative for an atrial fibrillation (211) over a time period of at least 1 minute,
      iii) a fibrillation mode termination criterion is fulfilled,
      iv) a fibrillation mode termination criterion has been fulfilled over a time period of at least 1 minute,
      switching the operational mode of the implantable medical device from either the atrial-fibrillation optimized long-term mode (225) or the confirmation phase (228) to a regular mode (229), wherein the regular mode (229) exclusively allows an atrial controlled stimulation of the right ventricle of the heart to be stimulated, wherein the regular mode (229) comprises activated additional functions that comprise the activated additional functions of the atrial-fibrillation optimized long-term mode (225) or of the confirmation phase (228).

1. Implantable medical device for stimulating a human or animal heart, comprising a processor, a memory unit, a stimulation unit configured to stimulate a human or animal heart, a first detection unit configured to detect an electric signal in an atrium of the same heart, and a second detection unit configured to detect an electric signal in a ventricle of the same heart,
wherein the memory unit comprises a computer-readable program that causes the processor to perform the following steps when executed on the processor:
a) repeatedly detecting, with the first detection unit, an atrial electric signal;
b) repeatedly determining whether the atrial electric signal is indicative for an atrial fibrillation;
c) if the atrial electric signal is indicative for an atrial fibrillation, switching an operational mode of the implantable medical device from a regular mode to a fibrillation mode, wherein the fibrillation mode exclusively allows a ventricular controlled stimulation of a ventricle of the heart to be stimulated, wherein at least one additional function that uses information from the right atrium, when the right atrium is not in an AF state, is deactivated;
and
switching the operational mode of the implantable medical device from the fibrillation mode to an atrial-fibrillation optimized long-term mode, if a switching criterion is fulfilled;
wherein the atrial-fibrillation optimized long-term mode exclusively allows a ventricular controlled stimulation of the ventricle of the heart to be stimulated, wherein the at least one additional function that was deactivated in the fibrillation mode is reactivated.

2. Implantable medical device according to claim 1, wherein the at least one additional function is chosen from the group comprising automatic pacing threshold monitoring, automatic sensing threshold monitoring, closed loop stimulation, beat-to-beat continuous capture control and automatic CRT-stimulation adaption.

3. Implantable medical device according to claim 1, wherein the switching criterion is at least one of the following criteria:
i) the atrial electric signal indicative for an atrial fibrillation is detected over a time period of at least 1 hour,
ii) the fibrillation mode is active over a time period of at least 1 hour,
iii) the fibrillation mode is active over a cumulative time period of at least 2 hours within 24 hours,
iv) the fibrillation mode was activated at least 120 (preferably 200) times within 24 hours.

4. Implantable medical device according to claim 1, wherein the program causes the processor to perform the following step after step d): switching the operational mode of the implantable medical device from the atrial-fibrillation optimized long-term mode to a confirmation phase, if at least one the following criteria is fulfilled:
i) the atrial electric signal is no longer indicative for an atrial fibrillation,
ii) the atrial electric signal has not been indicative for an atrial fibrillation over a time period of at least 1 minute to 30 minutes,
wherein the confirmation phase exclusively allows a ventricular controlled stimulation of the ventricle of the heart to be stimulated, wherein the confirmation phase comprises activated additional functions that correspond to the activated additional functions of the atrial-fibrillation optimized long-term mode, and
wherein the program causes the processor to perform an evaluate if a switch of the implantable device to the regular mode can be performed.

5. Implantable medical device according to claim 4, wherein the evaluate comprises an evaluation if among the last Y number of heart cycles, an X number of heart cycles occurred with an atrial heart rate below a threshold rate.

6. Implantable medical device according to claim 1, wherein the program causes the processor to perform the following step after step d): switching the operational mode of the implantable medical device from either the atrial-fibrillation optimized long-term mode or the confirmation phase to a regular mode, if at least one the following criteria i) and ii) and additionally at least one the following criteria iii) and iv) is fulfilled:
i) the atrial electric signal is no longer indicative for an atrial fibrillation,
ii) the atrial electric signal has not been indicative for an atrial fibrillation over a time period of at least 1 minute,
iii) a fibrillation mode termination criterion is fulfilled,
iv) a fibrillation mode termination criterion has been fulfilled over a time period of at least 1 minute,
wherein the regular mode exclusively allows an atrial controlled stimulation of the ventricle of the heart to be stimulated, wherein the regular mode comprises activated additional functions that comprise the activated additional functions of the atrial-fibrillation optimized long-term mode.

7. Implantable medical device according to claim 6, wherein the fibrillation mode termination criterion is a detected atrial electric signal that is indicative for an atrial rate higher than 80 bpm but lower than an atrial rate under atrial fibrillation.

8. Implantable medical device according to claim 1, wherein the ventricular controlled stimulation of the ventricle of the heart to be stimulated is an R-wave synchronous stimulation.

9. Implantable medical device according to claim 1, wherein the atrial controlled stimulation of the ventricle of the heart to be stimulated is a P-wave synchronous stimulation.

10. Implantable medical device according to claim 1, wherein the regular mode employs an atrial and a ventricular sensing, an atrial and/or a ventricular pacing and a dual control mode enabling pacing or inhibition of pacing in dependence on the sensed signals.

11. Implantable medical device according to claim 1, wherein the fibrillation mode employs i) an atrial and a ventricular sensing, an atrial and a ventricular pacing and an inhibiting control mode enabling inhibition of pacing or ii) a ventricular sensing, a ventricular pacing and an inhibiting control mode enabling inhibition of pacing.

12. Implantable medical device according to claims 1, wherein the atrial-fibrillation optimized long-term mode employs i) an atrial and a ventricular sensing, an atrial and a ventricular pacing and an inhibiting control mode enabling inhibition of pacing or ii) a ventricular sensing, a ventricular pacing and an inhibiting control mode enabling inhibition of pacing.

13. Implantable medical device according to claim 1, wherein the program causes the processor to reactivate all previously deactivated additional functions when switching the operational mode from the fibrillation mode to the atrial-fibrillation optimized long-term mode.

14. Method for controlling an implantable medical device according to claim 1, comprising the following steps:
a) repeatedly providing information on whether an atrial fibrillation is detected in a heart to be stimulated;
b) if an atrial fibrillation has been detected, switching an operational mode of the implantable medical device from an initial regular mode to a fibrillation mode, wherein the fibrillation mode exclusively allows a ventricular controlled stimulation of a ventricle of the heart to be stimulated, wherein at least one additional function that uses information from the right atrium, when the right atrium is not in an AF state, is deactivated; and
c) switching the operational mode of the implantable medical device from the fibrillation mode to an atrial-fibrillation optimized long-term mode, if a switching criterion is fulfilled;
wherein the atrial-fibrillation optimized long-term mode exclusively allows a ventricular controlled stimulation of the ventricle of the heart to be stimulated, wherein the at least one additional function that was deactivated in the fibrillation mode is reactivated.

15. Computer program product comprising computer-readable code that causes a processor to perform the following steps when executed on the processor:
a) repeatedly detecting, with a first detection unit of an implantable medical device for stimulating a human or animal heart, an atrial electric signal;
b) repeatedly determining whether the atrial electric signal is indicative for an atrial fibrillation;
c) if the atrial electric signal is indicative for an atrial fibrillation, switching an operational mode of the implantable medical device from an initial regular mode to a fibrillation mode, wherein the fibrillation mode exclusively allows a ventricular controlled stimulation of a ventricle of the heart to be stimulated, wherein at least one additional function that uses information from the right atrium, when the right atrium is not in an AF state, is deactivated;
switching the operational mode of the implantable medical device from the fibrillation mode to an atrial-fibrillation optimized long-term mode,
d) if a switching criterion is fulfilled,
wherein the atrial-fibrillation optimized long-term mode exclusively allows a ventricular controlled stimulation of the ventricle of the heart to be stimulated, wherein the at least one additional function that was deactivated in the fibrillation mode is reactivated.
